(19) United States
(12) Patent Application Publication
(10) Pub. No.: US 2005/0031762 A1
(43) Pub. Date: Feb. 10, 2005

(54) Low fat cocoa extract

(76) Inventors: James Gerard Mc Carthy, Noizay (FR); Hugues Brevard, Jongny (CH)

Correspondence Address: WINSTON & STRAWN;PATENT DEPARTMENT, 1400 L STREET, N.W., WASHINGTON, DC, 20005-3502, US

(21) Appl. No.: 10/914,008

(22) Filed: Aug. 6, 2004

Related U.S. Application Data
(63) Continuation of application No. PCT/EP03/02963, filed on Mar. 19, 2003.

(30) Foreign Application Priority Data
Mar. 20, 2002 (EP) 02006228.7

(51) Int. Cl. A23F 5/00

(52) U.S. Cl. 426/595

(57) ABSTRACT

The present invention relates to a low fat cocoa extract having a high level of cocoa flavors and cocoa flavor precursors, and a high level of antioxidants. It also relates to a cocoa flavor concentrate, an antioxidant concentrate, and a foodstuff containing them. It also relates to the use of in-vitro fermented beans as naturally fermented beans. The process used to obtain such extracts comprises the steps of placing cocoa seeds in acetic acid solution, heating the mixture, and concentrating the solids of the mixture after having removed the seeds.

### CROSS-REFERENCE TO RELATED APPLICATIONS

This application is a continuation of International application PCT/EP03/02963 filed Mar. 19, 2003, the entire content of which is expressly incorporated herein by reference thereto.

### FIELD OF THE INVENTION

The present invention relates to a low fat cocoa extract with high flavor potential and to the process for its preparation.

### BACKGROUND OF THE INVENTION

Cocoa seeds have to be fermented after their harvest. Fermentation is a key operation in developing a first-rate flavor. If the fermentation process is not well conducted, the flavor will be weak in intensity and often spoiled by off-flavors; without fermentation, flavor characteristics can be present in small amounts but are generally unusable for industrial purposes. There are several fermentation systems, depending on the cacao variety and the country of origin. Usually, 200-400 kg of beans, with the pulp, are piled on banana leaves, covered with more leaves and mixed every second day for a week. Special crates are also used: the beans in the crates are mixed every two days by transfer from one to another.

During the fermentation process, which usually lasts 3-6 days, several microorganisms develop naturally in the medium constituted by the heaps of beans and the surrounding pulp. During this period, the cocoa seeds lose their germinative power due to the breakdown of the cellular membranes, an event that also causes the cell contents to diffuse within the bean and thus become transformed enzymatically and chemically (Biehl, B. and Passer, D. (1982 J. Sci. Food Agric., 33, 1280-1290). These latter enzymatic and chemical changes include the production of cocoa flavor precursors and the reduction of negative attributes such as bitterness and astringency (“Industrial chocolate manufacture and use” by S. T. Beckett, third edition 1999, Blackwell Science, pp. 17-19).

Several methods have been described in the past to alter the cocoa flavor properties of cocoa beans or cocoa liquor. For example, U.S. Pat. No. 5,888,562 (equivalent to EP 7496949) discloses an enzymatic treatment of cocoa for overcoming the variability in the flavor precursor composition. To achieve this goal, liquor from unfermented or underfermented cocoa beans is incubated for 2 hours at 50° C. in water whose pH is adjusted to 4.5 with a solution of acetic acid to activate endogenous cocoa endoproteinase. Then, the mixture is submitted to another enzymatic treatment which is necessary to obtain flavor precursors. More particularly, a nib or liquor is prepared from cocoa beans fermented for 1 to 15 days, it is mixed with an aqueous medium at pH 3-6, the mixture is incubated at 40-60° C. for 10 minutes to 20 hours so as to promote the action of the cocoa endoprotease, the pH of the medium is adjusted to pH 4-8, at least one technical protease is added to it, and it is incubated at 10-60° C. for 5 minutes to 20 hours.

WO 00/22935 discloses a low-flavor cocoa obtained from unfermented cocoa beans by a two-step process, the first step consisting of destroying the cellular and subcellular structures by treatment with an aqueous acetic acid solution and the second step consisting of an oxidation treatment. This method suppresses the formation of flavor and hence low-flavor cocoa is obtained which is useful as substitute for cocoa butter, for example.

Kirchhoff et al, in an article published in 1989 in “Food Chemistry” (Vol. 31, pages 295-311), observed in a study of the “in-vitro fermentation” process that the acetic acid solution from the “in-vitro fermented” beans contained free amino acids, a significant proportion of which were hydrophobic amino acids.

There is a need for greater amounts of or enhanced cocoa flavor precursors, however, and these are now provided by the present invention.

### SUMMARY OF THE INVENTION

It has now been found that, surprisingly, the acid solution (leachate) from “in-vitro-fermented” beans contains a high level of cocoa flavor precursors, as well as high levels in cocoa antioxidants, such as polyphenols. Thus, the acetic acid extracts generated during the “in vitro fermentation” of beans can be used as a source of partially purified bio-active cocoa polyphenols and antioxidants, including leucocyanins, polyphenols, and the methylxanthines caffeine and theobromine, as well as a source of cocoa flavors and cocoa flavor precursors.

Accordingly, there is provided a low fat cocoa extract having a high level of cocoa flavor precursors and a high level of cocoa antioxidant compounds, as defined herein. According to other aspects of the invention, there is provided a cocoa flavor concentrate and/or an antioxidant concentrate and the use of such extracts as a source of antioxidants and/or cocoa flavor. A foodstuff can be enriched with either of these extracts. A process for producing the extract is disclosed, along with the use of extracts from such “in-vitro fermentations” to enhance cocoa flavor, especially at low levels. Furthermore, the invention concerns uses of the in-vitro fermented beans, and particularly the use of in-vitro fermented beans as a substitute for naturally fermented beans, to obtain products with enhanced flavor.

### DETAILED DESCRIPTION OF THE PREFERRED EMBODIMENTS

In the present description, by “dried acetic extract” and “extract”, it is understood an acetic acid extract from the “in-vitro fermentation” concentrated to a paste or to a dry mass, and by “in-vitro fermentation” it is understood treatment of cocoa beans processed in acetic acid for more than 4 hours.

By fresh seeds or cocoa beans it is understood beans or seeds freshly harvested from the cocoa pods and which have not been subjected to processing other than separation from the pulp. These beans are predominantly slaty and may have blue, purple or violet parts on their surface. By underfermented seeds or beans, it is understood seeds fermented for about 1 to 4 days. These beans are usually purple, blue and/or violet and may also be slaty, but not predominantly.

Lastly, by cocoa “flavor” it is understood cocoa flavor and flavor precursors.

All percentages are given by weight, unless otherwise specified.

The present invention includes a process where fresh seeds and/or underfermented seeds are placed in 100 mM acetic acid in autoclave bottles, in such a way that the solution completely covers the beans in the bottles. It is important for the purpose of this invention to use as a starting material fresh and/or underfermented coca beans or seeds that have not been submitted to drying or similar treatments. Preferred starting material consists in fresh beans. Then the bottles are placed in an incubator set at 40 to 55° C., preferably 50° C. for a period of time comprised between 4 and 80 hours, or more preferably 12-72 hours, or even more preferably 12-60 hours, in a preferred embodiment for 12-48 hours.

When seeds are removed, the viscous incubation solutions generated are stored at −20° C. Portions of this material can be thawed and the solids concentrated by evaporation, for example by rotary evaporation under vacuum at 50° C. The solids obtained, hereby called the “in-vitro fermentation” extract are used in various analysis and experiments described below. The seeds removed from the solution are placed in a hot air drier set at a temperature comprised between 50 and 70° C.

The extract obtained is a low fat cocoa extract (fat does not leach into the acetic acid solution), which surprisingly contains high levels of anti-oxidants and both cocoa flavors and cocoa flavor precursors. According to a first object of the invention, there is provided a low fat cocoa extract having a high level of cocoa flavor precursors and a high level of cocoa antioxidant compounds. The extract generally comprises solids having at least about 0.1% to 10% of cocoa flavors or flavor precursors comprising reactive free amino nitrogen containing compounds, and at least 1 to 20% of antioxidant compounds comprising polyphenols.

Cocoa flavor and cocoa flavor precursors containing amino groups were analyzed, in the “in-vitro fermented” solution, using the OPA method (Church F. et al, 1983, J. Dairy Science 66, 1219-1227) to determine the free amino nitrogen. Each of the acetic acid solutions are filtered and diluted, and the samples are reacted with 250 μL of the OPA solution. The mixes are allowed to react for 1 to 10 minutes, preferably 2 minutes, and then the absorbance is measured at 340 nm. The level of amino nitrogen containing molecules in the sample is calculated as leucine equivalents using an appropriate standard curve of different leucine concentrations reacted under the same conditions.

After a 48 hours incubation period, the “in-vitro fermentation” extract solution from beans of cacao variety CCN-51 contains from 2.74 to 3.1 mg/mL equivalents of leucine and the “in-vitro fermentation” extract solution from beans of the cacao variety EET-95 contains from 2.84 to 2.86 mg/mL equivalents of leucine. In the case of the CCN-51 solution, the solids content of the “in-vitro fermented” solution was determined to be 0.05 g/mL. Therefore, these results show that at least 5.6% of the solids in the CCN-51 acetic acid solution correspond to OPA reactive free amino nitrogen containing compounds such as the cocoa flavor precursors like aromatic amino acids and peptides. Generally an amount of at least 0.1 to 5% to as much as 6 to 7% to even as high as 10% of the solids correspond to such compounds.

SDS-PAGE gel analysis was performed to analyze the degradation of total cocoa bean proteins induced during the “in-vitro fermentation”. In order to determine the level of protein degradation induced during the “in-vitro fermentation”, acetone powders were made from these beans as described in Hansen et al. 1998 (J. Sci. Food Agric. 77, 273-281), after the pulp and testa have been removed.

The total proteins in this extract were then analyzed on a 10-20% Tris-Glycine SDS-PAGE gradient gel. The results showed that a significant amount of the bean proteins are degraded after 48 hours treatment. This is particularly clear for the two major vicilin proteins at approximately 48.5 and 34.1 kDa.

This analysis demonstrates that the acid incubation induced considerable protein degradation in the beans after 48 hours incubation, although it is less than that seen in a similar analysis of dried naturally fermented beans. Total polyphenol content of the 48 hour acetic acid extract was analyzed for the determination of polyphenol content. The spectrophotometric method (Folin-Ciocalteu Index) used to determine total polyphenol content closely corresponds to the method described in the Official Journal of European Communities, Chapter 41 (3.10. 1990, 178-179). CCN-51 48 Hour CCN-51 Extract Fresh Beans (dried solids) Total Polyphenols 32 mg ECE/g* ECE/g * mg ECE/g=milligrams of epicatechin equivalents per gram of sample. The results obtained show that the 48 hours acetic acid extract of CCN-51 beans contains 32 mg ECE/g of dried solids. Thus, polyphenols represent 3.2% of the dried solids in the extract. Assuming that the fresh CCN-51 beans are approximately 45% solids, this implies the polyphenols represent 5.48% of the dried solids in CCN-51 beans. This information demonstrates that the ratio of polyphenols to total solids in the 48 hour acetic acid extract of CCN-51 beans is approximately half that seen for the dried solids in unfermented CCN-51 beans.

In order to characterize the compounds in the extracts further, these “in-vitro fermentation” solutions were extracted with 80%/20% acetone/water. The 80%/20% acetone/water extracts of the acetic acid solutions obtained from the 48 hour “in-vitro fermented” EET-95 and CCN-51 beans, and the 80%/20% acetone/water extracts of fresh EET-95 and CCN-51 beans, were concentrated to dryness, solubilized in methanol and loaded on thin layer chromatography plates (TLC-0.25 mm silica pre-coated plates; Merck, 60-F254). After development with solvent A (ethyl acetate 65%, methanol 23%, and water 12%) and reaction with dihydroxy-1,3-naphtalene (which detects reducing compounds) one strong diffuse spot was seen in the acetic acid extracts of both cocoa varieties. This intense diffuse spot corresponds primarily to the carbohydrates in the pulp because the fresh beans, which were stripped of their pulp and testa, had much lower levels of this spot. Development with solvent A and reaction with FeCl3 (which detects phenolic compounds), indicated the presence of several similar spots in both the fresh beans and the concentrated acetic acid extract. This result shows that the phenolic composition of the acetic acid extracts are relatively similar to that seen of whole beans.

After development with solvent B (ethyl acetate 50%, methyl ethyl acetone 30%, formic acid 10%, and water 10%), a colored spot was seen for both the “in-vitro fermentation” extracts and the control whole bean extracts. This spot, which is seen without any treatment of the TLC plates, is due to the seed anthocyanins. Examination of this TLC plate with UV illuminated a spot with the same migration as theobromine in both the fresh bean material and the “in-vitro fermentation” extracts. When this plate was subsequently reacted with ninhydrin, two relatively strong spots appeared in all the extracts, and these spots are probably due to the presence of amino acids and small peptides in the fresh beans and in the “in-vitro fermentation” extracts. Overall, the results indicate that the acetic acid extracts from the “in-vitro fermentation” have many of the same acetone/water soluble compounds present in the fresh seeds, and thus the “in-vitro fermentation” extracts probably have a significant proportion of the flavor and antioxidant molecules present in the cacao seeds.

Preferably, the extract is an acetic acid extract that contains amino acids and peptides, as these are believed to be cocoa flavors and flavor precursors. To confirm the flavoring potential of these amino acids and peptides plus uncharacterized cocoa flavor precursors in the extracts, sniffing analysis of products formed after reacting the acetic acid extracts seeds of variety EET-95 and CCN-51 with fructose in a low water environment have been carried out. Before the reaction, the complete test mixes (extract, fructose, water, glycerol in ratio 1:1:1.5:96.5) had very little aroma and very little color. However, after 1 hour of reaction, both test mixes had developed aromas with strong cocoa and caramel notes.

Control experiments with lower fructose (0.1%) also had similar strong cocoa aromas. Other control reactions containing only 1% fructose, but with no acetic acid extract added, developed no cocoa aroma and had only a weak caramel aroma plus some clear off-notes. MS analysis of the products generated after a reaction of an acetic acid extract with sugar confirms that important cocoa aromas molecules, such as 2-and 3-methyl butanals, pentanedione, 2-methyl pyrazin, and phenylacetaldehyde are present in a heated reaction mix. Together, these results indicate that the 48 hour acetic acid extracts contain detectable cocoa aroma precursors.

In a preferred embodiment, the low fat cocoa extract contains flavors and/or flavor precursors which are present in an amount of at least 2 to 6%, more preferably 6 to 12%, and especially around 20%. The content of polyphenols present in the extract can vary according to, for example, the maturation of the seed when harvested, the variety of cocoa or the degree of purification. Nevertheless, the polyphenol content of the extract as disclosed here can be from at least 10 to 32 mg epicatechin equivalents/G extract, more preferably 30 to 100 mg epicatechin equivalents/g extract, and in a preferred embodiment, around 200 mg epicatechin equivalents/g extract. Preferably, the antioxidant compounds of the extract are bioactive.

According to another aspect of the invention, there is provided a cocoa flavor concentrate or a cocoa flavor concentrate which has been reacted with sugars. This concentrate contains cocoa flavors or flavor precursors including 2-and 3-methyl butanals, pentanedione, 2-methyl pyrazin, and phenylacetaldehyde.

The cocoa flavor concentrate obtained by this reaction method can be used directly as produced. The reaction allows at least part of the flavor precursors present in the concentrate to be transformed into cocoa flavors. The sugars which can be used for this reaction are well known by the skilled person, and it is possible to use, among others, sucrose, fructose, glucose, molasses, starch degradation products (glucose or maltose syrups, glucose-fructose syrups, polydextrose), milk sugars, fruit sugars (including levulose), sorbitol, xylitol or manitol, glycerol or a mixture thereof. The glycerol used in the reaction could also be replaced by polyethylene glycol or related solutions and fats such as milk fat, cocoa butter or vegetable fats, for example.

In this aspect of the invention, there is also provided an antioxidant concentrate obtainable by using an acetic acid extract from “in-vitro fermentation.” One way to obtain the antioxidant concentrate is to fractionate the original extract obtained from the “in-vitro fermentation” or to selectively purify it, by methods well known by those skilled in the art. This antioxidant concentrate can be used in any culinary products, as well as in the tires industry, the paintings industry, the pharmaceutical industry, or in cosmetics and health products, among others.

The expression “culinary product” is intended to encompass any consumable matter. Hence, it may be a product intended for the consumption by humans, but the term also encompasses products to be consumed by animals, for example pets, such as dogs, cats, rabbits, guinea pigs, mice, rats, birds (for example parrots), reptiles and fish (for example goldfish). However, the term also includes food to be consumed by other domesticated animals, such as livestock, for example, cattle, horses, pigs, sheep, goats, buffaloes, camels, and the like.

Another aspect of the invention concerns the use of the dried extract as a source of antioxidants and/or cocoa flavor. As described above, the dried acetic acid extract contains polyphenols and antioxidants, including leucocyanins, polyphenols, the methylxanthines caffeine and theobromine. Thus, the extract, or fractions thereof, can be used, for example, in dietary supplements as an anti-oxidant source, or in any manufactured food as an anti-oxidant source; it can be used in replacement of chocolate, for example in breakfast cereals, yoghurts or other dairy products, cakes, biscuits, cereal bars, coatings, drinks and beverages, sweets, powder chocolates, baby foods, jelly products, ice creams, toppings and sauces. It can also be used in any culinary product as defined above.

According to the a further aspect of the invention, there is provided a foodstuff enriched with the extract. Such foodstuff can be, for example, any culinary product as defined above, a dietary supplement, breakfast cereals, yogurts or other dairy products, cakes, biscuits, cereal bars, coatings, drinks and beverages, sweets, powder chocolates, baby foods, jelly products, ice creams, toppings and sauces, and more generally any type of manufactured food.

The foodstuff can be enriched with the extract, or fractions thereof. The extract, or fractions thereof, added to said foodstuff can be the acetic acid extracts of fresh or underfermented cocoa beans, but it can also be the previously described extract which has been subjected to reactions with sugars. It can also be a mixture of these two extracts.

When the foodstuff is enriched with an extract which has been submitted to a reaction with sugars, it allows said foodstuff to develop a full cocoa flavor.

The invention also relates to a process for obtaining an in-vitro fermentation extract comprising the steps of mixing cocoa seeds with acetic acid, heating the mixture, and concentrating the solids present in the liquid phase of the mixture for recovery as the extract. The heating step is preferably carried out at 40 to 55° C. for 4 to 72 hours, advantageously in autoclave bottles. The cocoa seeds can be fresh seeds or underfermented seeds, and the solids concentration can be out by rotary evaporation under vacuum. The resulting extract, wherein the cocoa flavors or flavor precursors are generally present in an amount of 0.1% to 20% of liquid extract, can be used as a cocoa flavor enhancer or as an antioxidant.

Another aspect of the invention concerns the use of an acetic acid extract from an “in-vitro fermentation” to enhance cocoa flavor. According to this aspect of the invention, the reacted dried acetic extract can be used in a preparation containing chocolate or chocolate extracts, as well as in preparations wherein the only chocolate source is the above-mentioned acetic acid extract, with or without sucrose, fructose, reducing sugars and/or other sweeteners.

The extract, reacted with sugars or not, can be added to the preparation at levels from 0.1% to 5.0%, when there is also another chocolate source, or at levels from 0.5% to 20% when there is no other chocolate source in the preparation.

Experiments have been performed to evaluate reacted dried acetic extracts to function as a cocoa flavor enhancer in a white chocolate model, at a level of 0.05% (final concentration). Various flavor reactions were carried out in two different low water environments at a concentration of 5% dried 48 hour CCN-51 acetic acid extract. The different low water. reaction backgrounds used were either propylene glycol (PG) or anhydrous milk fat. Subsequently, the flavor potential of the reaction flavors generated were evaluated in white chocolate at a 1% incorporation level. One other test, in which 1% of the dried 48 hour CCN-51 unreacted extract was incorporated directly into the white chocolate, was also carried out. Medium Ingredients Temp Time Incorporation in (° C.) (min) final chocolate product (%) * 92% PG, 1.5% 5% extract CCN51 125 60 1 § alkalized Water+1.5% Fructose 95% Anh. Milk 5% extract CCN51 125 60 1 fat No reaction pure extract CCN51 1 lyoph. (* 0.3% lecithin was also added in this sample). The products obtained were then tasted by an informal laboratory panel. The panel detected a weak cocoa flavor in the milk fat sample, and a weak cocoa like flavor in the sample with the 1% extract added.

Yet another aspect of the invention relates to the use of “in-vitro fermented” beans to replace naturally fermented beans. Indeed, “in-vitro fermented” beans have lost only part of their contents, such as part or all of their flavor precursors and antioxidants. They can, according to this aspect of the invention, be dried and used for the production of cocoa butter, coca liquor, cocoa powder or chocolate, among others, with the usual processing materials and standards. To further improve the flavor potential of these “in-vitro fermented” beans, they can for example be subjected to a short term natural fermentation and then dried either in the sun or by heating.

Thus, the invention relates to a method for producing cocoa butter, cocoa liquor, cocoa powder or chocolate products, the improvement which comprises substituting in-vitro fermented cocoa beans for fresh or unfermented beans to enhance cocoa flavor or antioxidant content of such products. If desired, the in-vitro fermented cocoa beans can be naturally fermented after being in-vitro fermented.

### EXAMPLES

The following examples are illustrative of some of the products and methods of making the same falling within the scope of the present invention. They are not to be considered in any way limitative of the invention. Changes and modifications can be made with respect to the invention. That is, the skilled person will recognize many variations in these examples to cover a wide range of formulas, ingredients, processing, and mixtures to rationally adjust the naturally occurring levels of the compounds of the invention for a variety of applications.

### Example 1

### Preparation of an In Vitro Fermentation Extract From Fresh Beans

Fresh beans from Theobroma cacao Trinitario ICS-95 are used in this example.

500 mL of 100 mM acetic acid are used to cover the beans in autoclave bottles.

The mixture of beans/acetic acid is incubated for 35 hours at 50° C., and the solids are then concentrated by rotary evaporation at 50° C. to give an “in-vitro fermentation” extract.

### Example 2

### Preparation of an In Vitro Fermentation Extract From Underfermented Beans

Underfermented beans from Theobroma cacao Trinitario ICS-95 are used in this example. These underfermented beans are 3-days harvested beans which have not been submitted to any treatment but have been left in an outside environment.

500 ML of 100 mM acetic acid are used to cover the beans in autoclave bottles.

The mixture beans/acetic acid is incubated for 39 hours at 53° C., and the solids are then concentrated by rotary evaporation at 50° C. to give an “in-vitro fermentation” extract.

### Example 3

### Preparation of a Chocolate Cake

The “in-vitro fermentation” extract of Examples 1 or 2 is used in dried form. The extract is added to a cake batter, according the following formulation (percentages in weight/weight of the final product): Shortening 11.4% Chocolate liquor 8.5% “In-vitro fermented” extract 0.5% Flour 22.9% Sugar 39.4% Baking powder 0.2% Salt 0.2% Eggs 16. 8% Aromas 0.1% The batter is then baked at 150° C. for 35 to 55 minutes, depending of the thickness of the cake.

### Example 4

### Preparation of a Chocolate Cake

The “in-vitro fermentation” extract of Examples 1 or 2 is used in dried form. The mixture is added to a cake batter, according the following formulation (percentages in weight/weight of the final product): Shortening 11.4% Cocoa powder 4.5% “in-vitro fermented extract 4.5% flour 22.9% Sugar 39.4% Baking powder 0.2% Salt 0.2% Eggs 16.8% Aromas 0.1% The batter is then cooked for 5 to 25 minutes at 190° C. depending on the thickness of the cake.

### Example 5

### Preparation of a Cake With Chocolate Flavor

The “in-vitro fermented” extract of Examples 1 or 2 mixture is added to a cake batter, according the following formulation (percentages in weight/weight of the final product): Shortening 16.2% “In-vitro fermented” extract 17% Flour 31.2% Sugar 16.6% Baking powder 0.3% Salt 0.1% Eggs 16.8% Aromas 0.1% Coloring 1.7% The batter is then cooked for 2 to 17 minutes at 220° C. depending on the thickness of the cake.

### Example 6

### Preparation of an Extruded Bar With High Levels of Polyphenols and Antioxidants

The “in-vitro fermented” extract of Examples 1 or 2 is added to the following preparation: Glucose-fructose syrup 20-30% Fruit/Fruit preparation 10-15% Protein powder 5-20% Micronutrients 4-5% Maltodextrin 10-15% Crisp cereal/cereal 10-33% In vitro fermented extract 5-20% Fat 2-5% Flavor 0.1-1.5% The ingredients are mixed until forming a homogenous mixture, under a temperature of 45° C. They are next extruded, the temperature of the extruding machine being set at 40° C. After extrusion, the bar is cooled at ambient temperature and packed.

### Example 7

### Preparation of Pizza Toasts

Toasts with pizza-like fillings are prepared according to the following formulation, wherein the “in-vitro fermentation” extract is obtained following Examples 1 or 2: 1.75 kg flour 0.02 kg sugar 0.02 kg milk proteins 10 g emulsifier 0.07 g bakery powder 0.06 g shortening 1.2 kg water 0.15 kg “in-vitro fermentation” extract The “in-vitro fermentation” extract is added as a source of antioxidants. The bread dough is knead, allowed to rest at 34° C. for 45 minutes, and cooked for 50 minutes at 220° C. The bread is then cut in 15 mm slices and the pizza-like filling, according to the following preparation, is put on the top of each bread slice: 80% chopped onions 5% olive oil 2% garlic in powdered form 8% tomato puree 0.8% salt 0.1% white pepper 0.1% chili pepper in powdered form 2% dried basil 2% “in-vitro fermentation” extract, as a source of antioxidants.

The proportion of filling, processed to have 70% water content, is set around 60% of the final toast weight.

### Example 8

### Preparation of a Milk Chocolate Bar

The “in-vitro fermentation” extract of Examples 1 or 2 is added to the following preparation (percentages in weight by weight of the final product): Chocolate liquor 8% Milk solids 20% Sugar 40% Cocoa butter 20% Water 1% “In-vitro fermented” extract 11% The “in-vitro fermented” extract is added at the beginning of the conching stage, in order to have a reaction time under heat conditions sufficient to develop the aromas of the cocoa flavor precursors in cocoa flavors. The addition of the “in-vitro fermented” extract allows this chocolate, poor in cocoa liquor, to have a full milk chocolate taste.

### Example 9

### Preparation of a Pet Food

An emulsion having the following composition is prepared: the “in-vitro fermented” extract being provided by following Examples 1 or 2 with a further treatment to eliminate theobromine from the extract (percentages given by weight): Chopped meat 60% Cereals 23% Plant proteins 2% Water 8% “In-vitro fermentation” extract 7% The “in-vitro fermentation” extract is used as a source of antioxidants and as a source of polyphenols.

### Example 10

### Preparation of a Body Lotion

A body lotion is prepared having the following composition, the “in-vitro fermented” extract being provided by the process of example 1 or example 2: Mineral oil 8.0% Isopropyl palmitate 5.0% Polyglyceryl-3-diiosostearate 2.0% Octyldodecanol 4.0% Carbomer 0.3% Cocoylglutamate sodium 0.2% Sodium hydroxide at 10% 1.2% Ascorbic acid 0.4% a-tocopherol 0.4% “In-vitro fermentation” extract 0.3% We then complete at 100% with water. The “in-vitro fermented” extract is incorporated to the body lotion as a source of antioxidants and polyphenols.

1. Low fat cocoa extract having a high level of cocoa flavors and/or flavor precursors and a high level of cocoa antioxidant compounds.

2. The extract of claim 1 comprising solids having at least about 0.1% to 10% of cocoa flavors or flavor precursors comprising reactive free amino nitrogen containing compounds, and at least 1 to 20% of antioxidant compounds comprising polyphenols.

3. The extract according to claim 1, wherein the extract is an acetic acid extract from an in-vitro fermentation of cocoa beans and the reactive free amino nitrogen compounds include aromatic amino acids or peptides.

4. The extract according to claim 2, wherein the cocoa beans are fresh beans or underfermented beans.

5. The extract according to claim 1 wherein the flavor precursors comprise leucine and the polyphenols are bioactive polyphenols that are present in an amount that provides at least 10 to 200 mg epicatechin equivalents/g liquid extract cocoa.

6. The extract according to claim 1, wherein the cocoa flavors or flavor precursors are present in an amount of 0.1% to 20% of liquid extract, and the polyphenols are present in an amount of about 30 to 200 mg epicatechin equivalents/g liquid extract.

7. A cocoa flavor concentrate obtainable from the extract of claim 1.

8. A cocoa flavor concentrate obtainable from the extract of claim 3.

9. The cocoa flavor concentrate according to claim 8, wherein the acetic acid extract is reacted with a sugar and contains cocoa flavors or flavor precursors including 2-and 3-methyl butanals, pentanedione, 2-methyl pyrazin, and phenylacetaldehyde.

10. An antioxidant concentrate obtainable by an in-vitro fermentation of fresh or underfermented cocoa beans.

11. A method for enhancing cocoa flavor which comprises adding to a foodstuff an acetic acid extract from an in-vitro fermentation of fresh or underfermented cocoa beans, with the extract added in an amount to enhance cocoa flavor available or derivable therefrom.

12. The method of claim 11 wherein the acetic acid extract is reacted with sucrose, fructose, reducing sugars or other sweeteners.

13. A foodstuff enriched with the extract of claim 1.

14. The foodstuff according to claim 13 in the form of a dietary supplement, breakfast cereal, yogurt, dairy product, cake, biscuit, cereal bar, coating, drink or beverage, sweet, powdered chocolate, baby food, jelly product, ice cream, topping or sauce.

15. A foodstuff enriched with the cocoa flavor concentrate of claim 7.

16. A foodstuff enriched with the cocoa flavor concentrate of claim 8.

17. A foodstuff enriched with the antioxidant concentrate of claim 10.

18. A process for obtaining an in-vitro fermentation extract comprising the steps of mixing cocoa seeds with acetic acid, heating the mixture, and concentrating the solids present in the liquid phase of the mixture for recovery as the extract.

19. The process according to claim 18 wherein the heating step is carried out at 40 to 55° C. for 4 to 72 hours.

20. The process according to claim 18, wherein the heating step is carried out in autoclave bottles.

21. The process according to claim 18 wherein cocoa seeds are fresh seeds or underfermented seeds.

22. The process according to claim 18, wherein the solids concentration is carried out by rotary evaporation under vacuum.

23. The process according to claim 18, wherein the extract is used as a cocoa flavor enhancer or as an antioxidant.

24. In a method for producing cocoa butter, cocoa liquor, cocoa powder or chocolate products, the improvement which comprises substituting in-vitro fermented cocoa beans for fresh or unfermented beans to enhance cocoa flavor or antioxidant content of such products.

25. The method of claim 24 wherein the in-vitro fermented cocoa beans are naturally fermented after being in-vitro fermented.
